(19) United States
(12) Patent Application Publication
(10) Pub. No.: US 2018/0055938 A1
(43) Pub. Date: Mar. 1, 2018

(54) Medication With Improved Taste And Sensory Experience

(71) Applicant: The Procter & Gamble Company, Cincinnati, OH (US)

(72) Inventors: Valerie Jean Naughton, Maineville, OH (US); Jason William Newlon, Lebanon, OH (US); Molly Findley, Cincinnati, OH (US)

(21) Appl. No.: 15/691,784

(22) Filed: Aug. 31, 2017

Related U.S. Application Data
(60) Provisional application No. 62/382,319, filed on Sep. 1, 2016.

(51) Int. Cl. A61K 47/18; A61K 31/09; A61K 31/137; A61K 31/485; A61K 31/167; A61K 9/08

(52) U.S. Cl. CPC A61K 47/18; A61K 31/09; A61K 9/08; A61K 31/485; A61K 31/167; A61K 31/137

(57) ABSTRACT

A liquid medication in a bottle that comprises three to four pharmaceutical actives selected from the group consisting of guaifenesin, acetaminophen, dextromethorphan, phenylephrine, pharmaceutically acceptable salts thereof and combinations thereof, ethyl 3-(p-menthane-3-carboxamido)acetate and menthol. The liquid medication can provide a taste and sensory experience that appeals to consumers.

### FIELD OF THE INVENTION

The present invention is directed towards a liquid medication, more particularly a liquid medication that provides a pleasant taste and sensory experience.

### BACKGROUND OF THE INVENTION

Liquid medications are routinely used by consumers to treat illness, mitigate symptoms, and/or to improve their health. However, many liquid medications have a sensory experience that is not desirable to consumers. Consumers have described the taste and mouth sensation of some medications as bitter, metallic, astringent, salty, numbing, stinging, burning, prickling, or irritating. These unpleasant tastes and/or sensations can be caused by some active ingredients and excipients.

To mitigate the negative aesthetics, liquid medications often contain flavors, sweeteners, and/or sensates to help improve the consumer's experience. However, despite these efforts, many liquid medications are rated poorly by consumers and fail to provide a sensory experience that consumers associate with an effective medicine.

Thus, there is a need for a liquid medication that gives consumers the perception that the medicine is strong and effective while still providing a pleasant flavor and balanced taste that consumers will accept.

### SUMMARY OF THE INVENTION

A liquid medication comprising: (a) three to four pharmaceutical actives selected from the group consisting of guaifenesin, acetaminophen, dextromethorphan, phenylephrine, pharmaceutically acceptable salts thereof and combinations thereof; (b) from about 0.01% to about 1% ethyl 3-(p-menthane-3-carboxamido)acetate by weight of the liquid medication; and (c) from about 0.01% to about 1% menthol by weight of the liquid medication; wherein the liquid medication is contained in a bottle.

A liquid medication comprising: (a) from about 1% to about 3% guaifenesin by weight of the liquid medication; (b) from about 0.01% to about 0.1% ethyl 3-(p-menthane-3-carboxamido)acetate by weight of the liquid medication; and (c) from about 0.01% to about 0.1% menthol by weight of the liquid medication; wherein the ratio of guaifenesin to ethyl 3-(p-menthane-3-carboxamido)acetate is from about 40:1 to about 80:1.

A personal health care array comprising: (a) a first liquid multi-symptom relief cold and flu product comprising pharmaceutical actives consisting of acetaminophen, dextromethorphan HBr, and phenylephrine HCl, and other sensory agents selected from the group consisting of N-ethyl-p-menthane-3-carboxamide, 3-1-menthoxy propane-1,2-diol, vanillyl butyl ether, 2-isopropyl-N,2,3-trimethylbutyramide, and combinations thereof; and (b) a second liquid multi-symptom relief cold and flu product comprising pharmaceutical actives consisting of guaifenesin, acetaminophen, dextromethorphan HBr, and phenylephrine HCl; about 0.01% to about 0.1% ethyl 3-(p-menthane-3-carboxamido)acetate by weight of the liquid medication; and about 0.03% to about 0.1% menthol by weight of the liquid medication.

### DETAILED DESCRIPTION OF THE INVENTION

Many liquid medications possess an unpleasant taste and/or after taste and fail to provide a desirable sensory experience. This can cause some consumers to avoid and/or dread taking liquid medications.

Guaifenesin is one of most bitter active pharmaceutical agents. It can be challenging to formulate liquid medications with a full dose of guaifenesin to cover the taste in a way that is acceptable to consumers. Menthol is a cooling sensate often used to try to mitigate the negative aesthetics of liquid medications. Although menthol can produce a physiological cooling effect that consumers enjoy, in some formulations menthol can exacerbate the bitterness of pharmaceutical actives like guaifenesin.

It has been found that, the combination of WS-5, menthol and flavoring agents can help cover the bitterness of the pharmaceutical actives and allow the cooling sensation of menthol to be elevated without causing burning.

It has also been found that products that offer a particular sensory experience receive a higher overall rating and give the perception that the medicine is powerful and effective. Consumer studies have shown that product ratings can be driven by a pleasant flavor and a balanced taste of sweetness and bitterness. In addition, consumers perceive a medicine to be effective when the product provides a balanced taste, an overall cooling and/or warming sensation, and throat coating.

By balancing sensory agents and flavoring agents, a liquid medication can be formulated to provide consumers with a sensory experience associated with effective medicine while still providing an acceptable taste, even in the presence of a full dose of bitter actives and/or excipients. In one example, the liquid medication can provide an increased cooling sensation and a stronger, more balanced flavor.

In one example, the liquid medication can comprise pharmaceutical actives selected from the group consisting of guaifenesin, acetaminophen, dextromethorphan, phenylephrine, pharmaceutically acceptable salts thereof, and combinations thereof, sensory agents, and flavoring agents. In one example, the sensory agents can be cooling sensates such as ethyl 3-(p-menthane-3-carboxamido)acetate (commercially available as “WS-5”) and menthol. In one example, the flavoring agent can be a citrus flavor. In one example the, the flavoring agent can be a berry flavor.

It has been found that, the combination of WS-5, menthol and flavoring agents can help cover the bitterness of the pharmaceutical actives and allow the cooling sensation of menthol to be elevated without causing burning.

As used herein, “dose” refers to a volume of liquid medication containing an amount of a pharmaceutical active suitable for administration on a single occasion, according to sound medical practice. A dose can be orally administered. In one example, a dose can be about 30 mL, in another example about 25 mL, in another example about 20 mL, in another example about 15 mL, and in another example about 10 mL. In another example, a dose of liquid medication can be from about 10 mL to about 75 mL, in another example from about 15 mL to about 50 mL, in another example from about 25 mL to about 40 mL, and in another example from about 28 mL to about 35 mL. The concentration of pharmaceutical actives can be adjusted to provide the proper doses of actives given the liquid dose size. In one example, the dose is intended to be administered every 4 hours, in another example every 6 hours, in another example every 8 hours, and in another example every 12 hours. As used herein, the articles “a” and “an” are understood to mean one or more of the material that is claimed or described, for example, “an active”.

All weights, measurements and concentrations herein are measured at 23 degrees Celsius (° C.) and 50% relative humidity, unless otherwise specified.

All percentages, parts and ratios are based upon the total weight of the liquid medication, unless otherwise specified. All such weights as they pertain to listed ingredients are based on the active level and, therefore, do not include carriers or by-products that may be included in commercially available materials.

### Sensory Test

A sensory test was conducted by a Descriptive Profile Panel (DPP) to understand the sensory attributes of liquid test formulations comprising either three pharmaceutical actives or four pharmaceutical actives. The DPP panel included up to 15 panelists that are trained and validated in Spectrum™ Descriptive Analysis methodology.

In the first phase of the test, each panelist received 30 mL of test samples having three pharmaceutical actives and a control, described hereafter. The panelists first assessed the aroma of the sample and then slurped the full dose into their mouth to assess the viscosity and in-mouth attributes. The panelists then swallowed the sample and assessed the after swallow attributes. Panelists measured each attribute at 4 time points (in-mouth, immediately after swallow, 3 minutes after swallow and 10 minutes after swallow) on a 0-60 scale for each time point, with the total attribute score recorded as the sum across all time points.

In the second phase of the test, the same panelists each received 30 mL of test samples having four pharmaceutical actives and a control, described hereafter. The panelists first assessed the aroma of the sample and then slurped the full dose into their mouth to assess the viscosity and in-mouth attributes. The panelists then swallowed the sample and assessed the after swallow attributes. Panelists measured each attribute at 4 time points (in-mouth, immediately after swallow, 3 minutes after swallow and 10 minutes after swallow) on a 0-60 scale for each time point, with the total attribute score recorded as the sum across all time points.

Table 1 describes the formulas of four of the samples ingested by the DPP Panelists.

TABLE 1

| | | Sample 2 | | Sample 4 |
|---|---|---|---|---|
| | Sample 1 | (Control) | Sample 3 | (Control) |
| | wt. % | wt. % | wt. % | wt. % |
| Propylene Glycol | 23.02 | 23.02 | 23.02 | 23.02 |
| Acetaminophen | 2.006 | 2.006 | 1.993 | 1.993 |
| Dextromethorphan | 0.062 | 0.062 | 0.061 | 0.061 |
| Hydrobromide (HBr) | | | | |
| Phenylephrine | 0.031 | 0.031 | 0.031 | 0.031 |
| Hydrochloride (HCl) | | | | |
| Guaifenesin | 0 | 0 | 1.227 | 1.227 |
| Menthol | 0.04 | 0 | 0.04 | 0.0046 |
| WS-5 | 0.015 | 0 | 0.03 | 0.0149 |
| Other Sensory Agents | 0 | 0.17 | 0 | 0.0010 |
| Flavor, citrus | 0.03 | 0.102 | 0 | 0 |
| Flavor, berry | 0 | 0 | 0.07 | 0.0699 |
| Xanthan Gum | 0.150 | 0.150 | 0.150 | 0.150 |
| Water Purified | 52.23 | 52.043 | 50.962 | 51.011 |
| Sodium benzoate | 0.100 | 0.100 | 0.100 | 0.100 |
| Sodium citrate dihydrate | 0.204 | 0.204 | 0.204 | 0.204 |
| Citric acid | 0.221 | 0.221 | 0.221 | 0.221 |
| Sodium saccharin | 0.100 | 0.100 | 0.100 | 0.100 |
| Sodium chloride | 0.500 | 0.500 | 0.500 | 0.500 |
| Sucralose | 0.07 | 0.07 | 0.07 | 0.07 |
| Color | 0.067 | 0.067 | 0.067 | 0.067 |
| Glycerin | 8.00 | 8.00 | 8.00 | 8.00 |
| Sorbitol | 13.154 | 13.154 | 13.154 | 13.154 |

The sensory results for the test samples evaluated did not differ significantly from one another. Table 2 summarizes the results for the three and four pharmaceutical active test samples and controls selected based on cost savings criteria. The numbers presented indicate the mean score from the panelists who graded each formula, with the total attribute score recorded as the sum across all time points.

TABLE 2
DPP Sensory Test Results

| | Mean Score by Example Number | | | |
|---|---|---|---|---|
| | 3 Active Formulations | | 4 Active Formulations | |
| | | Sample 2 | | Sample 4 |
| Sensory Attribute | Sample 1 | (Control) | Sample 3 | (Control) |
| Aroma Character | 12.71 | 4.70 | 17.06 | 16.49 |
| Total Cooling | 53.00 | 61.89 | 59.69 | 52.05 |
| After Swallow Vapors | 13.16 | 16.76 | 12.09 | 8.11 |
| Total Nasal Vapors | 20.17 | 24.33 | 17.44 | 12.75 |
| Total Throat Warming | 24.37 | 33.19 | 30.68 | 24.67 |
| Total Throat Cooling | 43.52 | 50.23 | 49.82 | 44.69 |

It was found that Sample 1, which contained 0.03% citrus flavor, 0.04% menthol and 0.015% WS-5, was rated lower for most of the sensory attributes evaluated, as compared to Sample 2, which contained 0.102% citrus flavor and 0.17% other sensory agents. In particular, the panelists found less total cooling, total throat warming, and total throat cooling in Sample 1 as compared to Sample 2. In addition, after swallow vapors and nasal vapors were decreased in Sample 1 as compared to Sample 2. However, Sample 1 was rated higher for aroma character as compared to Sample 2. It is believed that the decrease in total cooling, total throat warming and total throat cooling may be consumer noticeable.

It was found that Sample 3, which contained 0.07% berry flavor, 0.03% WS-5 and 0.04% menthol, was rated higher for most of the sensory attributes, as compared to Sample 4, which contained 0.0699% berry flavor, 0.0149% WS-5, 0.0046% menthol and 0.0010% other sensory agents. In particular, the panelists found more total cooling, total throat warming, and total throat cooling in Sample 3, as compared to Sample 4. The increase in total cooling, total throat warming and total throat cooling may be consumer noticeable. In addition, after swallow vapors and nasal vapors were increased in Sample 3 as compared to Sample 4. The aroma character was similar for Sample 3 and Sample 4.

### Consumer Study

Consumer studies were performed to understand the preferred sensory attributes and desired flavor character in liquid medication formulations.

Panelists were selected using the following criteria:
All suffered from a cough, cold, or flu in the past 12 months
All were treated with an over-the-counter medication in the past 12 months
All were liquid cough, cold, or flu medicine users or were open to using liquid cough, cold, or flu medicine.

In a first consumer study, commercially available liquid medication products were evaluated by consumers as follows.

A balanced incomplete block randomized sequential monadic blind show and taste test was performed. 285 consumer panelists used 8 of 20 commercially available liquid medication products, resulting in each product being used by 114 panelists. Each panelist swallowed 30 mL of the product and then filled out a questionnaire rating the product in terms of how much sensory attribute he or she noticed. The ratings were on a 100 point scale as follows: (100) excellent, (75) very good, (50) good, (25) fair and (0) poor. A waiting period of at least 6 hours was required between ingesting each product.

In a second consumer study, performed at a separate time, liquid test formulations having three pharmaceutical actives were evaluated by consumers as follows.

A randomized complete block sequential monadic of 7 test legs was performed. 100 consumer panelists swallowed 30 mL of each formulation tested. After swallowing, each consumer panelist filled out a questionnaire and rated the formulation in terms of how much sensory attribute he or she noticed in the formula. The ratings were on a 100 point scale as follows: (100) excellent, (75) very good, (50) good, (25) fair and (0) poor. The consumer panelists tested 2 formulations per day for 3 days, and 1 formula for 1 day. A waiting period of at least 4 hours was required between ingestion of each formulation.

In a third consumer study, performed at a separate time, liquid test formulations having four pharmaceutical actives were evaluated by consumers as follows.

A randomized complete block sequential monadic of 8 test legs was performed. A separate panel of 100 consumers swallowed 30 mL of each formulation tested. After swallowing, each consumer panelist filled out a questionnaire and rated the formulation as described above. The consumer panelists tested 2 formulations per day for 5 days. A waiting period of at least 4 hours was required between ingestion of each formulation.

Five of the commercially available products evaluated in the first consumer study are described in Table 3. The products presented represent the citrus-flavored three pharmaceutical active products and the four pharmaceutical active products evaluated.

TABLE 3

| | | Active | |
|---|---|---|---|
| | Dose | Ingredients | |
| Formulation | Size | per Dose | Excipients |
| TYLENOL ® | 15 mL | Acetaminophen | Alcohol, anhydrous |
| Cold Max | | (325 mg), | citric acid, FD&C |
| Daytime | | Dextromethorphan | yellow no. 6, |
| Citrus Burst | | HBr (10 mg), and | flavors, glycerin, |
| Liquid¹ | | Phenylephrine | propylene glycol, |
| | | HCl (5 mg) | purified water, sodium |
| | | | benzoate, sorbitol |
| | | | solution, sucralose |
| Walgreens ® | 15 mL | Acetaminophen | Butylated |
| Daytime | | (325 mg), | Hydroxyanisole, |
| Non-Drowsy | | Dextromethorphan | Edetate Disodium, |
| Cold & Flu | | HBr (10 mg), and | FD&C Yellow No. 6, |
| Relief² | | Phenylephrine | Flavor, Glycerin, |
| | | HCl (5 mg) | Menthol, Monobasic |
| | | | Sodium Phosphate, |
| | | | Polyethylene |
| | | | Glycol, Propylene |
| | | | Glycol, Purified |
| | | | Water, Saccharin |
| | | | Sodium, Sucrose, |
| | | | Xanthan Gum |
| Mucinex ® | 20 mL | Acetaminophen | Anhydrous citric acid, |
| Fast-Max ® | | (650 mg), | Edetate disodium, |
| Cold, | | Dextromethorphan | FD&C Blue #1, |
| Flu & Sore | | HBr (20 mg), | FD&C Red #40, |
| Throat³ | | Guaifenesin | Flavors, Glycerin, |
| | | (400 mg), and | Propylene glycol, |
| | | Phenylephrine | Propyl gallate, |
| | | HCl (10 mg) | Purified water, |
| | | | Sodium benzoate, |
| | | | Sorbitol, Sucralose, |
| | | | Trisodium citrate |
| | | | dihydrate, Xanthan |
| | | | gum |
| Tylenol ® | 15 mL | Acetaminophen | Anhydrous Citric |
| Severe | | (325 mg), | Acid, Ethyl Alcohol, |
| Daytime Cool | | Guaifenesin | FD&C Blue #1, |
| Burst ® Cold | | (200 mg), | Flavor, Glycerin, |
| Multi-Symptom⁴ | | Dextromethorphan | Propylene Glycol, |
| | | HBr (10 mg), and | Purified Water, |
| | | Phenylephrine | Sodium Benzoate, |
| | | HCl (5 mg) | Sorbitol Solution, |
| | | | Sucralose |
| TYLENOL ® | 15 mL | Acetaminophen | Anhydrous citric |
| Cold + Flu | | (325 mg), | acid, FD&C blue |
| Severe | | Guaifenesin | no. 1, FD&C red |
| Warming | | (200 mg), | no. 40, FD&C |
| Honey | | Dextromethorphan | yellow no. 6, |
| Lemon⁵ | | HBr (10 mg), | flavor, glycerin, |
| | | and Phenylephrine | propylene glycol, |
| | | HCl (5 mg) | purified water, sodium |
| | | | benzoate, sorbitol |
| | | | solution, sucralose |

¹Lot# ENCA; Expiration: November 2014; Purchased from CVS.com on May 16, 2013
²Lot# 3AK0721; Expiration: June 2014; Purchased from Walgreens.com on May 16, 2013
³Lot# 1002471; Expiration: October 2013
⁴Lot# DGCU; Expiration: June 2013
⁵Lot# FBCT; Expiration: January 2015; Purchased from CVS.com on May 16, 2013

Tables 4 and 5 summarize the results from the consumer tests of samples having three pharmaceutical actives (Consumer Study 1 and 2). Samples 1 and 2 presented in the tables below were not tested at the same time or by the same panel as the commercially available products; however, the data are shown together for ease of comparison.

Consumer tests were performed using similar test protocols and it is reasonable to believe that the results would not be significantly different even though different panels were used during the testing. The numbers indicate the average score from the consumer panelists who graded each formula.

TABLE 4

| | | | TYLENOL ® | Walgreens ® |
|---|---|---|---|---|
| | | | Cold Max | Daytime |
| | | | Daytime | Non-Drowsy |
| | Sam- | Sam- | Citrus Burst | Cold & Flu |
| Individual Attribute | ple 1 | ple 2 | Liquid | Relief |
| Overall (OA) Product | 62 | 57 | 53 | 59 |
| Rating | | | | |
| Perceived | 69 | 65 | 54 | 65 |
| Effectiveness | | | | |
| Thickness in Mouth | 68 | 63 | 51 | 65 |
| Ease of Swallowing | 76 | 63 | 69 | 63 |
| Aroma | 69 | 54 | 62 | 54 |
| OA Flavor | 58 | 49 | 50 | 48 |
| Flavor Strength | 60 | 55 | 54 | 54 |
| Sweetness | 60 | 47 | 52 | 47 |
| Bitterness | 55 | 46 | 45 | 43 |
| Medicinal Taste | 51 | 50 | 48 | 48 |
| Aftertaste | 58 | 44 | 50 | 42 |
| OA Cooling Sensation | 67 | 68 | 52 | 66 |
| OA Warming Sensation | 59 | 60 | 47 | 58 |
| Throat Coating | 62 | 63 | 54 | 66 |

TABLE 5

| | | | TYLENOL ® | Walgreens ® |
|---|---|---|---|---|
| | | | Cold Max | Daytime |
| | | | Daytime | Non-Drowsy |
| | Sam- | Sam- | Citrus Burst | Cold & Flu |
| Factor Averages | ple 1 | ple 2 | Liquid | Relief |
| OA Cooling/Warming & | 63 | 64 | 51 | 63 |
| Throat Coating | | | | |
| Pleasant Flavor | 62 | 53 | 56 | 52 |
| (Aroma, OA Flavor, | | | | |
| Flavor Strength) | | | | |
| Balanced Taste | 56 | 47 | 49 | 45 |
| (Sweet, Bitter, | | | | |
| Medicinal, Aftertaste) | | | | |

Doing a factor analysis, it was found that certain sensory attributes cluster together and drive overall consumer acceptance and the perception that the medicine is effective. It was found that consumers give an overall higher rating to products that provide a pleasant flavor and balanced taste. Pleasant flavor is the combination of aroma, overall flavor and flavor strength, while balanced taste is the combination of sweetness, bitterness, medicinal taste and aftertaste. If a liquid medication is too sweet, consumers believe that the medicine is formulated for children and may be less effective for adults. However, if a liquid medication is too bitter, consumers believe that the medicine may have too many side effects. It was also found that consumers perceive that a product is an effective medicine when it provides a sensation of overall cooling/warming and throat coating and also provides a balanced taste.

Sample 1, which was rated lower for cooling and warming sensory attributes according to the DPP panel, had a higher overall product rating and perception of effectiveness by consumers as compared to Sample 2. It was also found that consumers rated the overall cooling sensation of Sample 1 similar to Sample 2, even though the total level of sensory agents for Sample 1 was lower. In addition, consumers rated Sample 1 higher for pleasant flavor and balanced taste as compared to Sample 2, even though Sample 2 had a higher level of flavor and sensory agents.

When compared to commercially available citrus flavored liquid medications containing three pharmaceutical actives, consumers rated Sample 1 the highest for overall product rating, perception of effectiveness, pleasant flavor and balanced taste.

Tables 6 and 7 summarize the results from the consumer tests of samples having four pharmaceutical actives (Consumer Study 1 and 3). Samples 3 and 4 presented in the tables below were not tested at the same time or by the same panel as the commercially available products; however, the data are shown together for ease of comparison.

Consumer tests were performed using similar test protocols and it is reasonable to believe that the results would not be significantly different even though different panels were used during the testing. The numbers indicate the average score from the consumer panelists who graded each formula.

TABLE 6

| | | | | Tylenol ® | Tylenol ® |
|---|---|---|---|---|---|
| | | | | Severe | Cold and |
| | | | | Daytime | Flu |
| | | | Mucinex ® | Cool | Severe |
| | | | Fast Max ® | Burst ® | Warming |
| Individual | Sam- | Sam- | Cold, Flu & | Cold Multi- | Honey |
| Attributes | ple 3 | ple 4 | Sore Throat | Symptom | Lemon |
| OA Product | 58 | 53 | 49 | 46 | 31 |
| Rating | | | | | |
| Perceived | 63 | 59 | 49 | 55 | 44 |
| Effectiveness | | | | | |
| Thick in Mouth | 64 | 63 | 60 | 46 | 46 |
| Easy to Swallow | 69 | 62 | 62 | 57 | 58 |
| Aroma | 63 | 58 | 66 | 49 | 38 |
| OA Flavor | 50 | 44 | 40 | 34 | 23 |
| Flavor Strength | 53 | 50 | 47 | 45 | 33 |
| Sweetness | 51 | 43 | 44 | 36 | 27 |
| Bitterness | 46 | 42 | 35 | 40 | 31 |
| Medicinal Taste | 45 | 41 | 41 | 41 | 30 |
| Aftertaste | 46 | 40 | 34 | 34 | 24 |
| OA Cooling | 60 | 52 | 35 | 66 | 28 |
| Sensation | | | | | |
| OA Warming | 56 | 50 | 40 | 51 | 31 |
| Sensation | | | | | |
| Throat coating | 56 | 60 | 50 | 53 | 36 |

TABLE 7

| | | | | Tylenol ® | Tylenol ® |
|---|---|---|---|---|---|
| | | | | Severe | Cold and |
| | | | | Daytime | Flu |
| | | | Mucinex ® | Cool | Severe |
| | | | Fast Max ® | Burst ® | Warming |
| | Sam- | Sam- | Cold, Flu & | Cold Multi- | Honey |
| Factor Averages | ple 3 | ple 4 | Sore Throat | Symptom | Lemon |
| OA Cooling/ | 57 | 54 | 42 | 57 | 32 |
| Warming & | | | | | |
| Throat Coating | | | | | |
| Pleasant Flavor | 55 | 50 | 51 | 42 | 31 |
| (Aroma, OA | | | | | |
| Flavor, | | | | | |
| Flavor Strength) | | | | | |
| Balanced Taste | 47 | 42 | 39 | 38 | 28 |
| (Sweet, Bitter, | | | | | |
| Medicinal, | | | | | |
| Aftertaste) | | | | | |

It was found that Sample 3, which was rated higher for sensory attributes according to the DPP panel, had a higher overall product rating and perception of effectiveness as compared to Sample 4. In addition, consumers rated Sample 3 higher for overall cooling/warming and throat coating, pleasant flavor, and balanced taste as compared to Sample 4. Sample 3 had ten times more menthol and twice the amount of WS-5 as compared to Sample 4, which may have contributed to the higher level of bitterness, but still had a higher overall product rating.

When compared to commercially available liquid medications containing four pharmaceutical actives, Sample 3 had a similar sensation of overall cooling/warming and throat coating as Tylenol® Severe Daytime Cool Burst® Cold Multi-Symptom, but was rated higher than Mucinex® Fast Max® Cold, Flu & Sore Throat and Tylenol® Cold and Flu Severe Warming Honey Lemon. In addition, consumers rated Sample 3 the highest for overall product rating, perception of effectiveness, pleasant flavor and balanced taste.

Consumers prefer a liquid medication that provides a balanced taste and a sensory experience that matches the product's advertised benefits. In some examples, consumers may prefer a four pharmaceutical active product, which can be perceived as a stronger medicine, to provide a stronger sensory experience as compared to a three pharmaceutical active product. In other examples, consumers may prefer a three pharmaceutical active product to provide a stronger sensory experience as compared to a four pharmaceutical active product. In one example, consumers may prefer a liquid medication that provides a stronger aroma, overall flavor and/or flavor strength. In other examples, consumers may prefer a liquid medication with less aroma, overall flavor and/or flavor strength. In some examples, consumers may prefer a liquid medication with more sweetness, bitterness, and/or aftertaste. In other examples, consumers may prefer a liquid medication with less sweetness, bitterness, and/or aftertaste.

In one example, the liquid medication can comprise three pharmaceutical actives and can have a total cooling of greater than about 40 as determined by the DPP panel, in another example greater than about 50, and in another example greater than about 55. In one example, the liquid medication can comprise four pharmaceutical actives and can have a total cooling of greater than about 40 as determined by the DPP panel, in another example greater than about 45, in another example greater than about 50, and in another example greater than about 55. In one example, the liquid medication can comprise three pharmaceutical actives and can have a total cooling that is greater than the total cooling of a liquid medication comprising four pharmaceutical actives as determined by the DPP panel. In another example, the liquid medication can comprise three pharmaceutical actives and can have a total cooling that is less than the total cooling of a liquid medication comprising four pharmaceutical actives as determined by the DPP panel. In another example, the liquid medication can comprise three pharmaceutical actives and can have a total cooling that is approximately equal to the total cooling of a liquid medication comprising four pharmaceutical actives as determined by the DPP panel.

The liquid composition can comprise one or more sensory agents. Non-limiting examples of sensory agents can include cooling sensates, warming sensates, tingling sensates, and combinations thereof. Sensory agents can deliver sensory signals to the mouth, throat, nasal, and/or sinus passages so that the liquid medication may be perceived by the user as immediately acting to alleviate an ailment. In one example, sensory agents can work to provide the user with relief of symptoms by soothing the throat and/or giving the sensation of freer breathing.

Non-limiting examples of cooling sensates can include WS-23 (2-Isopropyl-N,2,3-trimethylbutyramide), WS-3 (N-ethyl-p-menthane-3-carboxamide), WS-30 (1-glyceryl-p-menthane-3-carboxylate), WS-4 (ethyleneglycol-p-methane-3-carboxylate), WS-14 (N-t-butyl-p-menthane-3-carboxamide), WS-12 (N-(4-,ethoxyphenyl)-p-menthane-3-carboxamide), WS-5 (ethyl 3-(p-menthane-3-carboxamido)acetate), menthol, 1-menthone glycerol ketal (sold as Frescolat® MGA by Symrise, Holzminden, Germany), (−)-Menthyl lactate (sold as Frescolat® ML by Symrise, Holzminden, Germany), (−)-Menthoxypropane-1,2-diol (sold as Coolact® 10 by Vantage Specialty Ingredients, Inc., Warren, N.J.), 3-(1-menthoxy)-2-methylpropane-1,2-diol, (−)-Isopulegol (sold as Coolact P® by Takasago International, Tokyo, Japan), cis & trans p-Menthane-3,8-diols (sold Coolact® 38D by Takasago International), menthyl pyrrolidone carboxylate (sold as Questice® by Givaudan Active Beauty, Verbuer, Switzerland), (1R,3R,4S)-3-menthyl-3,6-dioxaheptanoate (available from Firmenich, Geneva, Switzerland), (1R,2S,5R)-3-menthyl methoxyacetate (available from Firmenich), (1R,2S,5R)-3-menthyl 3,6,9-trioxadecanoate (available from Firmenich), (1R,2S,5R)-menthyl 11-hydroxy-3,6,9-trioxaundecanoate (available from Firmenich), (1R,2S,5R)-3-menthyl (2-hydroxyethoxy)acetate (available from Firmenich), Icilin also known as AG-3-5 (chemical name 1-(2-hydroxyphenyl)-4-(3-nitrophenyl)-3,6-dihydropyrimidin-2-one), 4-methyl-3-(1-pyrrolidinyl)-2[5H]-furanone, Peppermint oil, L-Monomenthyl succinate, L-monomenthyl glutarate, 2-1-menthoxyethanol (Coolact® 5), 3-1-Menthoxy propane-1,2-diol (sold as TK10 by Takasago International), N-(4-cyanomethylphenyl)-p-menthanecarboxamide (sold as Evercool™ 180 by Givaudan), and combinations thereof.

In one example, the liquid medication can comprise from about 0.001% to about 1% cooling sensate by weight of the liquid medication, in another example from about 0.01% to about 0.5% cooling sensate, in another example from about 0.02% to about 0.25%, and in another example from about 0.03% to about 0.10% cooling sensate.

In one example, the liquid medication can comprise from about 0.001% to about 1% WS-5 by weight of the liquid medication. In one example, the liquid medication can comprise from about 0.01% to about 0.8% WS-5, in another example from about 0.015% to about 0.5%, in another example from about 0.02% to about 0.25%, and in another example about 0.03% to about 0.15%. In one example, the liquid medication can comprise from about 0.01% to about 0.05% WS-5. In one example, the liquid medication can comprise about 0.03% WS-5. In one example, the liquid medication can comprise about 0.015% WS-5. One advantage to using WS-5 is that it may help with formulation stability. In one example, WS-5 is more soluble than WS-3. Another advantage to using WS-5 is that it can provide a long lasting, intense cooling sensation in the throat and oral cavity at a low level. Cooling sensates, such as WS-3 or 3-1-menthoxy propane-1,2-diol, may only provide a cooling sensation in the oral cavity or throat, respectively.

In one example, the liquid medication is substantially free of WS-3. As used herein, substantially free of WS-3 means that the liquid composition comprises less than about 0.1%, alternatively less than about 0.05%, alternatively less than about 0.01%, alternatively less than about 0.001% WS-3, by weight of the liquid composition.

In one example, the liquid medication does not comprise 3-1-menthoxy propane-1,2-diol because it can increase the bitterness of guaifenesin.

In one example, WS-5 can provide stronger cooling properties and/or a longer lasting cooling sensation than WS-3, WS-12, and WS-23.

In one example, the liquid medication can comprise from about 0.001% to about 1% menthol by weight of the liquid medication. In one example, the liquid medication can comprise from about 0.01 to about 0.5%, in another example from about 0.02% to about 0.25%, and in another example about 0.03% to about 0.1%. In one example, the liquid medication can comprise about 0.04% menthol, and in another example about 0.05% menthol. In one example, menthol may exert a nasal decongestion effect, cough suppression, oral anesthetic, and/or antitussive action. In one example, the liquid medication does not comprise greater than about 0.04% menthol because higher levels of menthol may increase the bitterness of liquid formulations containing guaifenesin.

In one example, the liquid medication can contain only one cooling sensate and in another example the liquid medication can contain more than one cooling sensate. In one example, the liquid medication can comprise both WS-5 and menthol as cooling sensates. In one example, the ratio of menthol to WS-5 can be from about 1:1 to about 4:1, in another example about 1.2:1 to about 3:1, and in another example from about 1.3:1 to about 2.6:1.

Non-limiting examples of warming sensates can include vanillyl alcohol n-butyl ether (sold as TK-1000 by Takasago International), Heatenol™ (available from Sensient Pharmaceutical, St. Louis, Mo.), Optaheat (sold by Symrise, Holzminden, Germany), ginger extract, capsicum tincture, cinnamon, capsaicin, curry, Isobutavan, Nonivamide, vanillyl butyl ether (commercially available as Hotact® VBE), piperine, and combinations thereof. Warming sensates can be present from about 0.005% to about 2% by weight of the liquid medication, in another example from about 0.01% to about 1%, and in another example from about 0.1% to about 0.5%.

Non-limiting examples of tingling sensates can include sichuan pepper, hydroxy alpha sanshool, jambu extracts, spilanthol, and combinations thereof. In one example, tingling sensates can be present from about 0.005% to about 1% by weight of the liquid medication, in another example from about 0.01% to about 0.5%, and another example from about 0.015% to about 0.3%.

The composition can optionally comprise one or more salivation agents. Non-limiting examples of salivation agents include formula (I):

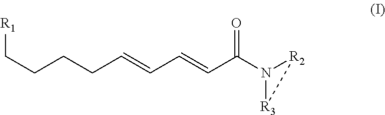

wherein R₁ represents C1-C2 n-alkyl; R₂ is 2-methyl-1-propyl and R₃ is hydrogen, or R₂ and R₃ taken together is a moiety (designated by the dashed lines) having the formula —(CH₂)ₙ— wherein n is 4 or 5, and combinations thereof.

In an embodiment, the salivating agent comprises a material wherein R₂ is 2-methyl-1-propyl and R₃ is hydrogen, in another embodiment the salivating agent comprises a material wherein R₁ is C1 n-alkyl, R₂ is 2-methyl-1-propyl and R₃ is hydrogen. In another embodiment, the salivating agent comprises trans-pellitorin, a chemical having a structure according to formula (II):

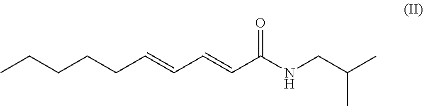 [SMILES: CCCCC/C=C/C=C/C(=O)NCC(C)C]

In another embodiment, the salivation agent can comprise sodium bicarbonate, sodium chloride, trans-pellitorin, and combinations thereof. In one example, salivation agents can be present from about 0.05% to about 2% by weight of the liquid medication, in another embodiment from about 0.1% to about 1%, and in another example from about 0.25% to about 0.75%.

In one example, the liquid medication may comprise one or more active pharmaceutical agents. In one example, the liquid medication may comprise three or more active pharmaceutical agents. In one example, the liquid medication comprises three active pharmaceutical agents. In another example, the liquid medication comprises four active pharmaceutical agents.

In one example, the active pharmaceutical agent is a multi-symptom relief (MSR) cold/flu active which can be used to treat one or more cold/flu symptoms. MSR cold/flu actives can be used to treat a variety of cold/flu symptoms including nasal congestion, runny nose, sneezing, headache, dry cough, sore throat, sinus pressure or pain, chest congestion, muscle aches/pains, wet/chesty cough, fever, and combinations thereof. MSR cold/flu actives can include decongestants, expectorants, antihistamines, antitussives, pain relievers, and combinations thereof.

Non-limiting examples of expectorants can include guaifenesin, ambroxol, bromhexine, and combinations thereof. In one example, the expectorant can be guaifenesin.

In one example, a dose can comprise about 50 mg of guaifenesin, in another example about 100 mg, in another example about 200 mg, and in another example about 400 mg. In one example, the liquid medication can comprise from about 0.1% to about 6% guaifenesin by weight of the liquid medication, in another example about 0.5% to about 4%, and in another example about 1% to about 3%.

In one example, the liquid medication can comprise guaifenesin and WS-5, and the ratio of guaifenesin to WS-5 can be from about 10:1 to about 1,000:1, in another example from about 20:1 to about 100:1, and in another example from about 40:1 to about 80:1.

Non-limiting examples of antihistamines can include chlorpheniramine, desloratadine, levocetirizine, diphenhydramine, doxylamine succinate, triprolidine, clemastine, pheniramine, brompheniramine, dexbrompheniramine, loratadine, cetirizine and fexofenadine, amlexanox, alkylamine derivatives, cromolyn, acrivastine, ibudilast, bamipine, ketotifen, nedocromil, omalizumab, dimethindene, oxatomide, pemirolast, pyrrobutamine, pentigetide, thenaldine, picumast, tolpropamine, ramatroban, repirinast, suplatast tosylate aminoalkylethers, tazanolast, bromodiphenhydramine, tranilast, carbinoxamine, traxanox, chlorphenoxamine, diphenylpyaline, embramine, p-methyldiphenhydramine, moxastine, orphenadrine, phenyltoloxamine, setastine, ethylenediamine derivatives, chloropyramine, chlorothen, methapyrilene, pyrilamine, talastine, thenyldiamine, thonzylamine hydrochloride, tripelennamine, piperazines, chlorcyclizine, clocinizine, homochlorcyclizine, hydroxyzine, tricyclics, phenothiazines, mequitazine, promethazine, thiazinamium methylsulfate, azatadine, cyproheptadine, deptropine, desloratadine, isothipendyl, olopatadine, rupatadine, antazoline, astemizole, azelastine, bepotastine, clemizole, ebastine, emedastine, epinastine, levocabastine, mebhydroline, mizolastine, phenindamine, terfenadine, tritoqualine, phenylephrine (PE), pseudophedrine (PSE) and combinations thereof.

In one example, the liquid medication can comprise from about 0.01% to about 0.1% antihistamine by weight of the liquid medication, in another example from about 0.02% to about 0.07% antihistamine, and in another example from about 0.03% to about 0.05% antihistamine.

In one example, the antihistamine can be PE and pharmaceutically acceptable salts thereof. In one example, a dose of the liquid medication can contain about 2.5 mg PE, in another example, about 5 mg PE, in another example about 10 mg PE, and in another example about 20 mg PE. Illustrative salts of PE include phenylephrine hydrochloride and phenylephrine hydrobromide.

In another example, the antihistamine can be PSE. In one example, a dose of the liquid medication can contain about 120 mg PSE and in another example about 30 mg PSE. In one example, the antihistamine can be doxylamine succinate and a dose of the liquid medication can contain about 12.5 mg doxylamine succinate. In another example, the antihistamine can be chlorpheniramine. In one example, a dose of the liquid medication can contain about 2 mg of chlorpheniramine and in another example a dose of the liquid medication can contain about 4 mg of chlorpheniramine.

Non-limiting examples of antitussives can include dextromethorphan (DXM), codeine, chlophedianol, levodropropizine, and combinations thereof. In one example the liquid medication can comprise from about 0.01% to about 0.2% antitussive by weight of the liquid medication, in another example from about 0.025% to about 0.1%, and in another example from about 0.04% to about 0.075% antitussive.

In one example, the antitussive is DXM and pharmaceutically acceptable salts thereof, such as dextromethorphan hydrobromide. In one example a dose of liquid medication can comprise about 5 mg DXM, in another example about 10 mg DXM, in another example about 15 mg DXM, in another example about 20 mg DXM, and in another example about 30 mg DXM.

Non-limiting examples of pain relievers can include acetaminophen (APAP), ibuprofen, ketoprofen, diclofenac, naproxen, aspirin, and combinations thereof. In one example, the liquid medication can comprise from about 0.5% to about 3.5% pain reliever by weight of the liquid medication, in another example from about 1% to about 3% pain reliever, and in another example from about 1.5% to about 2% pain reliever. In one example the pain reliever is APAP. In one example, the liquid medication can comprise about 80 mg APAP, in another example about 160 mg, in another example about 325 mg, and in another example about 650 mg.

In one example, the liquid medication can contain water and propylene glycol. In one example, the liquid medication can comprise from about 15% to about 80% water, in another example from about 25% to about 75% water, in another example from about 40% to about 70% water, in another example from about 35% to about 65% water, and in another example from about 45% to about 60% water. In another example, the liquid medication can contain from about 5% to about 40% propylene glycol, in another example from about 15% to about 35% propylene glycol, and in another example from about 20% to about 30% propylene glycol.

In one example, the liquid medication can comprise from about 1% to about 15% ethanol, in another example from about 3% to about 12% ethanol, and in another example from about 6% to about 10% ethanol.

The liquid medication can comprise a sweetener to provide sweetness and taste masking of the actives and excipients that provide a bitter character. In one example, the liquid medication can comprise from about 2% to about 25% sweetener, in another example from about 5% to about 20% sweetener, in another example from about 7% to about 15% sweetener, and in another example from about 8% to about 12% sweetener.

Non-limiting examples of sweeteners can include nutritive sweeteners, sugar alcohols, synthetic sugars, high intensity natural sweeteners, and combinations thereof. Non-limiting examples of nutritive sweeteners can include fructose, galactose, and combinations thereof. In one example, the sweetener can be high fructose corn syrup. Non-limiting examples of sugar alcohols can include xylitol, sorbitol, mannitol, maltitol, lactitol, isomalt, erthritol, glycerin, and combinations thereof. In one example, the liquid medication can comprise from about 1% to about 30% sugar alcohol, in another example from about 5% to about 28% sugar alcohol, in another example about 10% to about 25% sugar alcohol, and in another example about 13% to about 23% sugar alcohol. In one example, the liquid medication can comprise from about 5% to about 20% sorbitol, in another example from about 7% to about 18% sorbitol, and in another example from about 10% to about 15% sorbitol. In another example, the liquid medication can comprise from about 3% to about 15% glycerin, in another example from about 5% to about 10% glycerin, and in another example from about 7% to about 9% glycerin.

Non-limiting examples of synthetic sweeteners can include sodium saccharin, acesulfame potassium, sucralose, aspartame, neohesperidin dihydrochalcone, neotame, cyclamates, and mixtures thereof. In one example, the liquid medication can comprise from about 0.01% to about 0.5% artificial sweetener, in another example from about 0.1% to about 0.3% artificial sweetener, and in another example about 0.15% to about 0.25% artificial sweetener.

Non-limiting examples of high intensity natural sweeteners can include stevioside, rebaudioside A, rebaudioside C, dulcoside, monoammonium glycrrhizinate, thaumatin, and combinations thereof.

In one example, the liquid medication can comprise a buffer. The buffer can help maintain a constant pH within the liquid medication. In one example, the liquid medication can comprise from about 0.05% to about 2% buffer, in another example from about 0.1% to about 1% buffer, in another example from about 0.15% to about 0.5% buffer, and in another example from about 0.18% to about 0.25% buffer. Non-limiting examples of buffers can include acetic acid, sodium acetate, citric acid, sodium citrate, monobasic sodium phosphate, dibasic sodium phosphate, sodium carbonate, sodium bicarbonate, succinic acid, sodium succinate, potassium dihydrogen phosphate, and phosphoric acid.

In one example, the liquid medication can comprise a preservative. In one example the liquid medication can comprise from about 0.01% to about 1% preservative, in another example from about 0.05% to about 0.5% preservative, in another example from about 0.07% to about 0.3% preservative, and in another example from about 0.08% to about 0.15% preservative. Non-limiting examples of preservatives can include benzalkonium chloride, ethylenediaminetetraacetic acid (EDTA), benzyl alcohol, potassium sorbate, parabens, benzoic acid, sodium benzoate, and mixtures thereof.

In one example, the liquid medication can comprise a thickener. In one example, the liquid medication can comprise from about 0.01% to about 3% thickener, in another example about 0.05% to about 1.5% thickener, in another example about 0.1% to about 0.75% thickener, and in another example about 0.12% to about 0.3% thickener. Non-limiting examples of thickeners can include xanthan gum, carrageenan, polyacrylic acid, polyvinylpyrrolidone, cellulosic polymers including carboxymethycellulose, hydroxethylcellulose, hydroxymethylcellulose, and hydroxypropylmethylcellulose, and combinations thereof.

The liquid medication can be any color. Non-limiting examples of colors can include red, green, amber, orange, yellow, blue, pink, violet, turquoise, and combinations thereof. In one example, the liquid medication is green. In another example, the liquid medication is clear.

The liquid medication can comprise a dye that provides the color. Non-limiting examples dyes that may be used in the present invention include FD&C blue #1, FD&C blue #2, D&C blue #4, D&C blue #9, FD&C green #3, D&C green #5, D&C green #6, D&C green #8, D&C orange #4, D&C orange #5, D&C orange #10, D&C orange #11, FD&C red #3, FD&C red #4, D&C red #6, D&C red #7, D&C red #17, D&C red #21, D&C red #22, D&C red #27, D&C red #28, D&C red #30, D&C red #31, D&C red #33, D&C red #34, D&C red #36, D&C red #39, FD&C red #40, D&C violet #2, FD&C yellow #5, FD&C yellow #6, D&C yellow #7, Ext. D&C yellow #7, D&C yellow #8, D&C yellow #10, D&C yellow #11, and combinations thereof. In one example, the liquid medication can comprise from about 0.001% to about 0.1% dye, in another example from about 0.002% to about 0.05% dye, and in another example from about 0.003% to about 0.01% dye.

The liquid medication can comprise a flavoring agent. Non-limiting examples of flavoring agents can include natural flavoring agents, artificial flavoring agents, artificial extracts, natural extracts and combination thereof. Non-limiting examples of flavoring agents can include: vanilla, honey lemon cherry vanilla, apple, yumberry, mangosteen, peach, honey ginger, chamomile, cherry, cherry cream, mint, vanilla mint, dark berry, black berry, raspberry, peppermint, spearmint, honey peach, acai berry, cranberry, honey cranberry, tropical fruit, dragon fruit, wolf berry, red stem mint, pomegranate, black currant, strawberry, lemon, lime, peach ginger, orange, orange cream, creamsicle, apricot, anethole, ginger, jack fruit, star fruit, blueberry, fruit punch, lemon grass, chamomile lemon grass, lavender, banana, strawberry banana, grape, blue raspberry, lemon lime, coffee, espresso, cappuccino, honey, wintergreen mint, bubble gum, tart honey lemon, sour lemon, green apple, boysenberry, rhubarb, strawberry rhubarb, persimmon, green tea, black tea, red tea, white tea, honey lime, cherry lime, apple, tangerine, grapefruit, kiwi, pear, vanillin, ethyl vanillin, maltol, ethyl-maltol, pumpkin, carrot cake, white chocolate raspberry, chocolate, white chocolate, milk chocolate, dark chocolate, chocolate marshmallow, apple pie, cinnamon, hazelnut, almond, cream, crème Brule, caramel, caramel nut, butter, butter toffee, caramel toffee, aloe Vera, whiskey, rum, cocoa, licorice, pineapple, guava, melon, watermelon, elderberry, raspberries and cream, peach mango, cool berry, lemon ice, nectar, spicy nectar, tropical mango, apple butter, peanut butter, tangerine, tangerine lime, marshmallow, cotton candy, apple cider, orange chocolate, and mixtures thereof.

The liquid medication can comprise from about 0.01% to about 1% flavoring agent, in another example from about 0.03% to about 0.5%, and in another example from about 0.1% to about 0.3%.

In one example, the liquid medication can comprise a citrus flavoring agent. In one example, the liquid medication can comprise a berry flavoring agent.

In one example, the liquid medication can be sold and/or stored in a primary container. In one example, the primary container can be a bottle for containing a liquid. In one example, the primary container can be translucent. In one example, the primary container can be opaque.

The primary container can be made out of any suitable material. Non-limiting examples of suitable materials for the primary container can include glass, polyethylene terephthalate (PET), Glycol-modified Polyethylene Terephthalate (PETG), Oriented Polypropylene (OPP), Polyvinylchloride (PVC), Polyvinylidene Chloride (PVDC), Nylon, Polyethylene Terphthalate Polyester (PETP), Polyphene, and combinations thereof. In one example, the primary container can be made out of PET.

In one example, a primary container can contain any number of unit doses. In one example, the primary container contains one unit dose. In one example, the primary container can comprise unit doses that are intended to be consumed from 2 to 8 hours, in another example 4 to 8 hours, in another example 8 to 16 hours, in another example from 12 to 24 hours, in another example from 2 to 14 days, in another example from 3 to 12 days, in another example 4 to 10 days, in another example 5 to 9 days, in another example 6 to 8 days, and in another example 7 days. How the doses are intended to be consumed can be determined by package directions or guidance from the manufacturer, government health regulatory body or United States Federal Drug Administration (FDA). In one example, the directions can be printed on the primary container, printed on a secondary container, or can otherwise accompany the primary container, such as a package insert. In one example, the primary package comprises from 1 fluid ounce (fl. oz.) (0.03 L) to 24 fl. oz. (0.71 L), in another example from 1.5 fl. oz (0.04 L) to 18 fl. oz. (0.53 L), in another example 2 fl. oz. (0.06 L) to 16 fl. oz. (0.47 L), in another example 3 fl. oz. (0.9 L) to 12 fl. oz. (0.35 L), in another example 4 fl. oz. (0.1 L) to 10 fl. oz. (0.30 L), and in another example 6 fl. oz. (0.17 L) to 8 fl. oz. (0.24 L).

The primary container can be of varying shape and size as desired based upon the number, size and type of unit doses contained therein. In one example, the primary container can be sized to be conveniently portable.

In one example, the primary container can be packaged in a secondary package. The secondary container can be made from a variety of materials, non-limiting examples of which include paper, cardboard, paperboard, clear wrap, sleeve, plastic and combinations thereof. In one example, there is no secondary container.

In one example, a consumer can go to a store because she has a cold and/or flu and needs one or more products to help alleviate her symptoms. The consumer can locate an array of cold and/or flu products on a shelf in the store. In one example, the array can comprise a first liquid multi-symptom relief cold and flu product comprising three pharmaceutical actives adjacent or proximal to a second liquid multi-symptom relief cold and flu product comprising four pharmaceutical actives on the shelf. In one example, the first liquid product can comprise acetaminophen, dextromethorphan HBr, and phenylephrine HCl. In one example, the second liquid product can comprise guaifenesin, acetaminophen, dextromethorphan HBr, and phenylephrine HCl.

In one example, the first liquid product can comprise less WS-5 than the second liquid product. In one example, first liquid product can comprise about half the amount of WS-5 than the second liquid product. In one example, the first liquid product and the second liquid product can have about equal amounts of WS-5.

In one example, the first liquid product can comprise other sensory agents selected from the group consisting of N-ethyl-p-menthane-3-carboxamide, 3-1-menthoxy propane-1,2-diol, vanillyl butyl ether, 2-isopropyl-N,2,3-trimethylbutyramide, and combinations thereof. In one example, the first liquid product is substantially free of WS-5. As used herein, substantially free of WS-5 means that the first liquid product comprises less than about 0.1%, alternatively less than about 0.05%, alternatively less than about 0.01%, alternatively less than about 0.001% WS-5, by weight of the liquid composition.

In one example, the second liquid product is substantially free of vanillyl butyl ether. In one example, the only sensory agents in the second liquid product are menthol and WS-5.

In one example, the first liquid product can comprise less menthol than the second liquid product. In one example, the first liquid product can comprise about half the amount of menthol than the second liquid product. In one example, the first liquid product and the second liquid product can comprise about equal amounts of menthol. In one example, the first liquid product does not contain menthol.

In one example, the first liquid product can optionally comprise less than about 0.03% WS-5 and the second liquid product can comprise from about 0.01% to about 0.20% WS-5.

In one example, products can be formulated for daytime use. In one example the products can be formulated for nighttime use. In one example, the liquid medication can comprise instructions that direct a user to ingest the medication at night before bedtime.

In one example, the array is located inside the store near the pharmacy, in another example it is located inside the store near the checkout, in another example the array is movable and can change locations either inside the store or outside the store. In one example, the array is located at the end of the aisle in an end cap. The array can take up the entire end cap or it can take up a portion of the end cap. In another example, the array is located in an aisle; it can be at an end of the aisle or in the middle of an aisle. In one example, the array can be a free-standing kiosk that can be located in an open area so consumers can reach it from multiple directions. In another example, the array is able to easily be moved to different areas of the store.

The liquid medication can be packaged with other items as part of a personal health care kit. In one example, the kit can comprise a first liquid multi-symptom relief cold and flu product and a second liquid multi-symptom relief cold and flu product.
Samples 1-2 were made as follows. First, a glycol premix was made by putting propylene glycol in a container and beginning agitation. Then APAP, DXM, menthol, WS-5, other sensory agents, and flavoring agents (if present in the formulation) were added to the glycol premix and mixed until all of the components were fully dissolved. Then the thickener, xanthan gum, was added and mixed until homogenous and the final premix was made.

Then, a main mix was made by adding purified water to a container and beginning agitation. Then the buffer salts, which included sodium citrate dihydrate and citric acid, were added and mixed until dissolved. Then the sodium saccharin, sucralose, color, sodium benzoate and PE were added and mixed until dissolved to make the main mix.

Next, the final premix was added to the main mix and the final premix vessel was rinsed with purified water and added to the main mix. Then the sorbitol and glycerin were added to the mixture and it was mixed until fully homogeneous.

Samples 3-4 were made as follows. First, a glycol premix was made by putting propylene glycol in a container and beginning agitation. Then APAP, DXM, GG, menthol, WS-5, other sensory agents, and flavoring agents (if present in the formulation) were added to the glycol premix and mixed until all of the components were fully dissolved. Then the thickener, xanthan gum, was added and mixed until homogenous and the final premix was made.

Then, a main mix was made by adding purified water to a container and beginning agitation. Then the buffer salts, which included sodium citrate dihydrate and citric acid, were added and mixed until dissolved. Then the sodium saccharin, sucralose, color, sodium benzoate and the PE were added and mixed until dissolved to make the main mix.

Next, the final premix was added to the main mix and the final premix vessel was rinsed with purified water and added to the main mix. Then the sorbitol and glycerin were added to the mixture and it was mixed until fully homogeneous.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A liquid medication comprising:
a. three to four pharmaceutical actives selected from the group consisting of guaifenesin, acetaminophen, dextromethorphan, phenylephrine, pharmaceutically acceptable salts thereof and combinations thereof;
b. from about 0.01% to about 1% ethyl 3-(p-menthane-3-carboxamido)acetate by weight of the liquid medication; and
c. from about 0.01% to about 1% menthol by weight of the liquid medication;
wherein the liquid medication is contained in a bottle.

2. The liquid medication of claim 1 wherein the pharmaceutical actives consist of acetaminophen, dextromethorphan HBr, and phenylephrine HCl.

3. The liquid medication of claim 1 wherein the pharmaceutical actives consist of guaifenesin, acetaminophen, dextromethorphan HBr, and phenylephrine HCl.

4. The liquid medication of claim 1 wherein the pharmaceutical actives comprise guaifenesin and wherein a dose of the liquid medication comprises about 200 mg guaifenesin.

5. The liquid medication of claim 1 wherein the pharmaceutical actives comprise guaifenesin and wherein a dose of the liquid medication comprises about 400 mg guaifenesin.

6. The liquid medication of claim 1 wherein the ratio of menthol to ethyl 3-(p-menthane-3-carboxamido)acetate is from about 1.2:1 to about 3:1.

7. The liquid medication of claim 1 comprising about 0.04% menthol by weight of the liquid medication.

8. The liquid medication of claim 1 wherein the liquid medication is substantially free of N-ethyl-p-menthane-3-carboxamide.

9. The liquid medication of claim 2 comprising about 0.015% ethyl 3-(p-menthane-3-carboxamido)acetate by weight of the liquid medication.

10. The liquid medication of claim 3 comprising about 0.03% ethyl 3-(p-menthane-3-carboxamido)acetate by weight of the liquid medication.

11. A liquid medication comprising:
a. from about 1% to about 3% guaifenesin by weight of the liquid medication;
b. from about 0.01% to about 0.1% ethyl 3-(p-menthane-3-carboxamido)acetate by weight of the liquid medication; and
c. from about 0.01% to about 0.1% menthol by weight of the liquid medication;
wherein the ratio of guaifenesin to ethyl 3-(p-menthane-3-carboxamido)acetate is from about 40:1 to about 80:1.

12. The liquid medication of claim 11 further comprising acetaminophen, dextromethorphan HBr, and phenylephrine HCl.

13. The liquid medication of claim 11 comprising about 0.03% ethyl 3-(p-menthane-3-carboxamido)acetate by weight of the liquid medication.

14. The liquid medication of claim 11 comprising from about 0.04% menthol by weight of the liquid medication.

15. A method of treating cold and flu symptoms in a human in need thereof comprising administering the liquid composition of claim 11.

16. The liquid medication of claim 11 wherein the liquid medication is adapted for consumption during the day.

17. A personal health care array comprising:
a. a first liquid multi-symptom relief cold and flu product comprising pharmaceutical actives consisting of acetaminophen, dextromethorphan HBr, and phenylephrine HCl; and
other sensory agents selected from the group consisting of N-ethyl-p-menthane-3-carboxamide, 3-1-menthoxy propane-1,2-diol, vanillyl butyl ether, 2-isopropyl-N,2,3-trimethylbutyramide, and combinations thereof; and
b. a second liquid multi-symptom relief cold and flu product comprising pharmaceutical actives consisting of guaifenesin, acetaminophen, dextromethorphan HBr, and phenylephrine HCl;
about 0.01% to about 0.1% ethyl 3-(p-menthane-3-carboxamido)acetate by weight of the liquid medication; and
about 0.03% to about 0.1% menthol by weight of the liquid medication.

18. The personal health care array of claim 17 wherein the first liquid multi-symptom relief cold and flu product is substantially free of ethyl 3-(p-menthane-3-carboxamido)acetate.

19. The personal health care array of claim 17 wherein the second liquid multi-symptom relief cold and flu product comprises about 0.03% ethyl 3-(p-menthane-3-carboxamido)acetate by weight of the liquid medication.

20. The personal health care array of claim 17 wherein the second liquid multi-symptom relief cold and flu product comprises a ratio of menthol to ethyl 3-(p-menthane-3-carboxamido)acetate of about 2.6:1.
